(19) United States
(12) Patent Application Publication
(10) Pub. No.: US 2008/0208035 A1
(43) Pub. Date: Aug. 28, 2008

(54) Device for superposed MRI and PET imaging

(76) Inventors: Jurgen Nistler, Erlangen (DE); Wolfgang Renz, Erlangen (DE); Lothar Schon, Neunkirchen (DE); Stefan Stocker, Grossenseebach (DE)

Correspondence Address: HARNESS, DICKEY & PIERCE, P.L.C., P.O.BOX 8910, RESTON, VA, 20195, US

(21) Appl. No.: 12/071,104

(22) Filed: Feb. 15, 2008

(30) Foreign Application Priority Data
Feb. 26, 2007 (DE) 10 2007 009 184.4

(51) Int. Cl. A61B 5/055

(52) U.S. Cl. 600/411

(57) ABSTRACT

A device is disclosed for superposed magnetic resonance imaging and positron emission tomography imaging. The device includes a magnetic resonance imaging magnet which defines a longitudinal axis; a magnetic resonance imaging gradient coil arranged radially within the magnetic resonance imaging magnet; a magnetic resonance imaging RF coil arranged radially within the magnetic resonance imaging gradient coil; and a multiplicity of positron emission tomography detection units arranged in pairs opposite one another about the longitudinal axis. In at least one embodiment, the many positron emission tomography detection units are arranged radially within the magnetic resonance imaging gradient coil and can be inserted into the device and can be removed from the device along the longitudinal axis. In at least one embodiment, a carrier tube is provided having a multiplicity of pockets, which in each case extend along the longitudinal axis, for accommodating at least one positron emission tomography detection unit.

### PRIORITY STATEMENT

The present application hereby claims priority under 35 U.S.C. §119 on German patent application number DE 10 2007 009 184.4 filed Feb. 26, 2007, the entire contents of which is hereby incorporated herein by reference.

### FIELD

Embodiments of the present application generally relate to a device for superposed MRI and PET imaging.

### BACKGROUND

It is well known that a magnetic resonance imaging unit (MRI unit) comprises inter alia those three functional assemblies which are illustrated in FIG. 5: a basic field system 21, a gradient system 22 and a radio frequency system 23 (also referred to as an RF system or body resonator). In general, the basic field system 21 is a magnet providing a strong, static magnetic field. The gradient system provides a variable magnetic field in the low frequency range up to approximately 1 kHz which has a linearly increasing or decreasing profile in one or more directions. The RF system 23 provides an oscillating magnetic field in the radio frequency range for deflection of the nuclear spins, at the nuclear spin resonant frequency (in general, 42.45 MHz) governed essentially by the static magnetic field, which can furthermore also serve for receiving signals from the relaxing nuclear spins.

In conventional magnetic resonance imaging units, these three assemblies are arranged around the patient to be examined in the radial direction in the following order, starting from the inside and moving outward: RF system 23, gradient system 22 and basic field system 21. The patient is placed on a couch 24 located radially inside the RF system 23.

In addition to magnetic resonance imaging (MRI), positron emission tomography (PET) has also become more popular for medicinal diagnosis in recent years. Whereas MRI is an imaging method for displaying structures and slice images within the body, PET allows visualization and quantification of metabolic activities in-vivo.

PET uses the particular properties of positron emitters and of positron annihilation to quantitatively determine the function of organs or cell areas. In this case, appropriate radiopharmaceuticals marked with radionuclides are administered to the patient before the examination. The radionuclides emit positrons as they decay, which interact with an electron after a short distance, resulting in so-called annihilation. This creates two gamma quanta, which fly apart in opposite directions (offset by 180°). The gamma quanta are detected by two mutually opposite PET detector modules within a specific timeframe (coincidence measurement), from which the location of the annihilation is determined at a point on a connecting line between these two PET detector modules.

For detection purposes, the PET detector modules are arranged annularly around the patient and in general cover the majority of the arc length of the gantry. Each PET detector module generates an event record on detecting a gamma quantum, which indicates the time and the detection location, that is to say the corresponding detector element. These items of information are transferred to fast logic and compared. If two events occur within a maximum specified time interval, a gamma decay event is assumed to have occurred on the connecting line between the two associated PET detector modules. The PET image is reconstructed by a tomography algorithm, that is to say the so-called back projection.

In many cases, superposed, imaging of the MRI and PET methods is desirable due to the different information acquired by the two methods.

Combining the imaging MRI and PET methods into one appliance means that the two units of the RF system and the PET detectors, which are required for data acquisition, must be arranged within the basic field system and the gradient system. A concentric arrangement, in which the RF system would be positioned within the annularly arranged PET detectors, has to date been fraught with difficulties.

Firstly, the structure of the internal coil arrangement (emitting and receiving coils) of the RF system reduces the sensitivity of the annularly arranged PET detectors, requiring correction during the PET image reconstruction.

Furthermore, the internal diameter remaining for the patient is greatly reduced by the interleaving, from the inside outward, of the RF system and the annularly arranged PET detectors.

Moreover, the spacing required for a high quality of the RF body resonator between the annularly arranged PET detectors and the RF conductor structures must be greatly reduced (reverse field flux space).

Finally, there is no space available for shielding (for example, by septa) the annularly arranged PET detectors from gamma radiation from outside of the annularly arranged PET detectors due to the spatial conditions in the radial direction.

For example, this can be solved by high levels of integration or interlocking of the RF system and the PET detectors, leading to greater mechanical complexity and work in the event of a fault. Another solution essentially dispenses with integration, as illustrated in a comparative example in FIG. 6. This results in comparatively large spatial requirements, however. Reference symbol 22 refers to the gradient system 22. Reference symbol 23 refers to the RF system with reverse field flux space. Reference symbol 25 refers to annularly arranged PET detection units. Reference symbol 26 refers to an installation gap between the gradient system 22 and a carrier tube 27 for the PET detection units. Reference symbol 28 refers to an RF shield and reference symbol 29 refers to interior cladding or a carrier tube for the RF system 23.

### SUMMARY

In at least one embodiment of the present invention, a device is provided for superposed MRI and PET imaging which restricts the internal diameter of the patient tunnel less but still provides shielding and an excellent image quality.

According to at least one embodiment of the present invention, a device for superposed magnetic resonance imaging and positron emission tomography imaging has a magnetic resonance imaging magnet which defines a longitudinal axis; a magnetic resonance imaging gradient coil arranged radially within the magnetic resonance imaging magnet; a magnetic resonance imaging RF coil arranged radially within the magnetic resonance imaging gradient coil; and a multiplicity of positron emission tomography detection units arranged in pairs opposite one another about the longitudinal axis. The many positron emission tomography detection units are arranged radially within the magnetic resonance imaging gradient coil and can be inserted into the device and removed from the device along the longitudinal axis. The maintenance of the device is thus greatly simplified, since the PET detection units can easily be removed along the longitudinal axis.

Furthermore, a carrier tube is preferably provided having a multiplicity of pockets which in each case extend along the longitudinal axis for accommodating at least one positron emission tomography detection unit.

The inventive device has the following advantages:

(a) The positron emission tomography detection units including the electronics are arranged such that they can be removed individually along the longitudinal axis and without involved disassembly of the fittings arranged radially on the outside.
(b) There is only a minimal spatial requirement for a concentric arrangement and simultaneous separation of the magnetic resonance imaging RF coil and positron emission tomography detection units.
(c) The positron emission tomography detection units are preferably located in their own carrier tube, thus allowing replacement of a single detector by pulling out in the z direction.
(d) The attenuation of the gamma radiation by structures located within the PET ring is reduced to a minimum by a thin tube wall in the vicinity of the PET crystals.
(e) Simple cooling of the positron emission tomography detection units is made possible by the carrier tube. “Simple” water cooling or air cooling can be used since it is located outside the RF transmitting antenna, avoiding convolution artifacts in the MRI image.
(f) The carrier tube allows high stiffness, resulting in a reduction in mechanical deformation and noise transmission by the gradient coil.
(g) The MR and PET signals can be “decoupled” to a great extent due to multiple shielding, that is to say by the RF shield and the shielding structure in the pockets for the positron emission tomography detection units.

### BRIEF DESCRIPTION OF THE DRAWINGS

Example embodiments of the invention are now described with reference to the attached drawings, in which

FIG. 1 shows a device for superposed magnetic resonance imaging and positron emission tomography imaging according to a first example embodiment of the present invention;

FIG. 2 shows a longitudinal sectional view of the section illustrated in FIG. 1 of the device for superposed magnetic resonance imaging and positron emission tomography imaging according to the first example embodiment of the present invention;

FIG. 3 shows a device for superposed magnetic resonance imaging and positron emission tomography imaging according to a second example embodiment of the present invention;

FIG. 4 shows a device for superposed magnetic resonance imaging and positron emission tomography imaging according to a third example embodiment of the present invention;

FIG. 5 shows a known device for magnetic resonance imaging according to the prior art; and

FIG. 6 shows a comparative example of a device for superposed magnetic resonance imaging and positron emission tomography imaging.

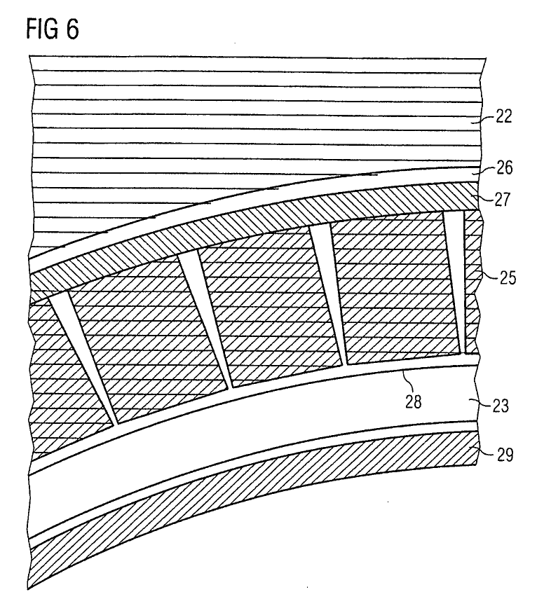

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described. Like numbers refer to like elements throughout. As used herein, the terms “and/or” and “at least one of” include any and all combinations of one or more of the associated listed items.

The example embodiments of the present invention are described in the following text with reference to the drawings.

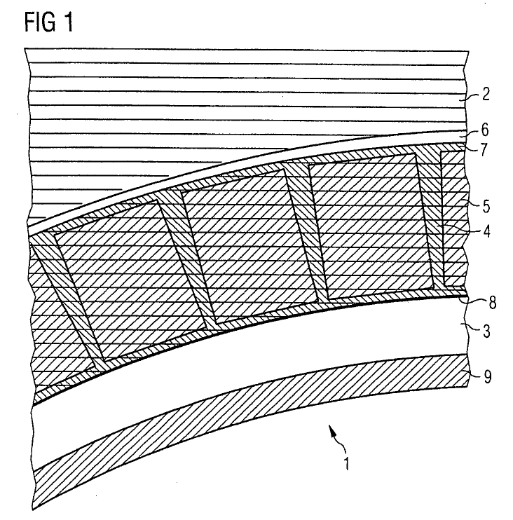

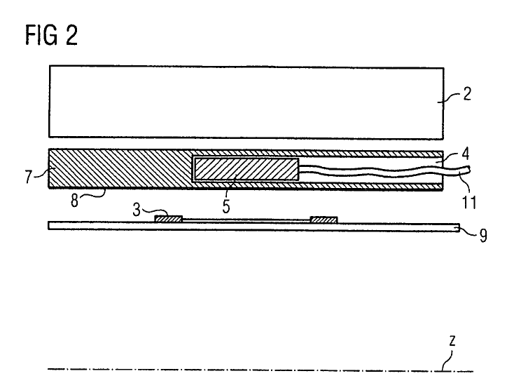

FIG. 1 shows a device 1 for superposed magnetic resonance imaging and positron emission tomography imaging according to the first example embodiment of the present invention, and FIG. 2 shows a longitudinal sectional view of a section of the device 1 for superposed magnetic resonance imaging and positron emission tomography imaging according to the first example embodiment of the present invention shown in FIG. 1.

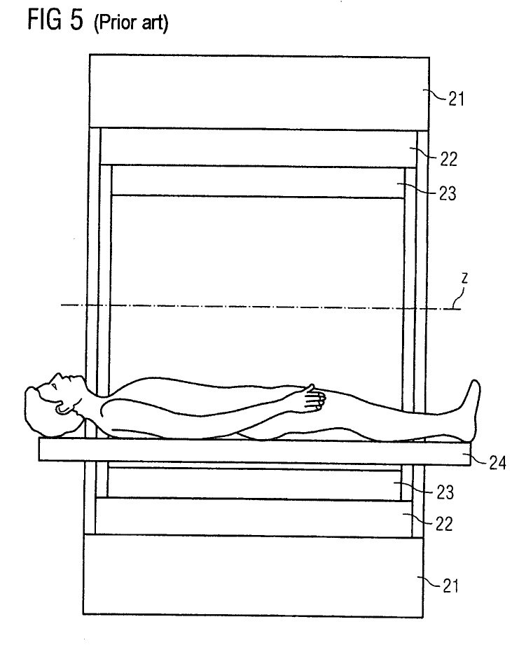

According to an example embodiment of the present invention, the device 1 for superposed magnetic resonance imaging and positron emission tomography imaging has a magnetic resonance imaging magnet (not illustrated) which defines a longitudinal axis z, as illustrated in FIG. 5. The magnetic resonance imaging magnet forms a basic field system, which provides a strong, static magnetic field.

Furthermore, the device 1 includes a magnetic resonance imaging gradient coil 2, arranged radially inside the magnetic resonance imaging magnet and preferably coaxially to the longitudinal axis z. The magnetic resonance imaging gradient coil 2 forms a gradient system, which provides a low-frequency variable magnetic field.

Moreover, the device 1 includes a magnetic resonance imaging RF coil 3, arranged radially inside the magnetic resonance imaging gradient coil 2 and preferably coaxially to the longitudinal axis z. The magnetic resonance imaging RF coil 3 forms an RF system, which provides an oscillating magnetic field in the radio frequency range for deflection of the nuclear spins at the nuclear spin resonant frequency (for example, approximately 42.58 MHz at 1 T, 63.87 MHz at 1.5 T or 127.74 MHz at 3 T) governed essentially by the static magnetic field. Furthermore, the magnetic resonance imaging RF coil 3 can also serve for receiving signals from the relaxing nuclear spins.

Furthermore, the device 1 has a multiplicity of positron emission tomography detection units 5 arranged in pairs opposite one another about the longitudinal axis z. The many positron emission tomography detection units 5 are arranged radially inside the magnetic resonance imaging gradient coil 2 and preferably coaxially to the longitudinal axis z.

The positron emission tomography detection units 5 in each case have an avalanche photodiode array with a lutetium oxyorthosilicate crystal array and an electrical amplifier circuit upstream thereof, which greatly assists the compact embodiment of the positron emission tomography detection units 5. However, the invention is not limited to the use of the avalanche photodiode array with the lutetium oxyorthosilicate crystal array and the electrical amplifier circuit upstream thereof. Different compact positron emission tomography detection units can be used as well.

The reference symbol 6 refers to a gap between the magnetic resonance imaging gradient coil 2 and a carrier tube 7 for the PET detection units 5. The reference symbol 8 refers to an RF shield, fitted to the inside of the carrier tube 7. The reference symbol 9 refers to internal cladding or a carrier tube for a transmitting antenna of the RF system 3.

According to an embodiment of the invention, the many positron emission tomography detection units 5 can be inserted into the device 1 and can be removed from the device 1 along the longitudinal axis z. The carrier tube has a multiplicity of pockets 4 (see FIG. 2) which in each case extend along the longitudinal axis z for accommodating at least one positron emission tomography detection unit 5.

This saves space in the radial direction in the device 1, which leads to the advantages listed in the introduction to the description.

Preferably, a metal layer is applied to the inner faces of the pockets 4, and serves for shielding and heat conduction. In particular, thin metallization, which can be applied in the pockets 4, serves as additional shielding for the positron emission tomography detection units 5 and prevents the radiation of interference.

FIG. 2 indicates how the many positron emission tomography detection units 5 can be inserted into the device and can be removed from the device along the longitudinal axis z. The pockets 4 preferably simultaneously serve as a cable feed for the positron emission tomography detection units 5. The positron emission tomography detection units 5 can advantageously be pulled out of the pockets by cables 11 attached to them.

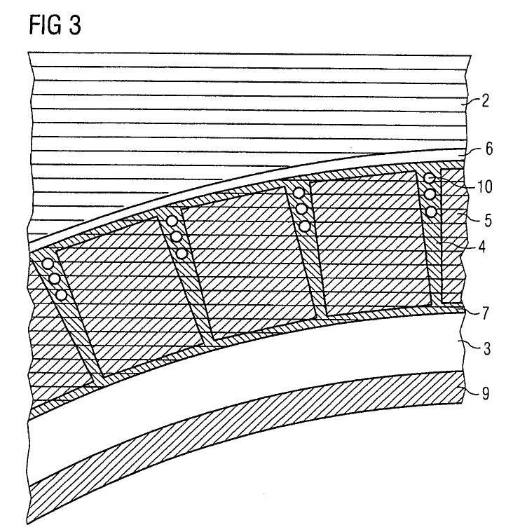

FIG. 3 shows a device for superposed magnetic resonance imaging and positron emission tomography imaging according to the second example embodiment of the present invention.

In the second example embodiment, cooling channels 10 are provided between two adjacent pockets 4 for carrying a coolant, such as water, air or any other cooling fluid.

The carrier tube 7, which is preferably produced using an encapsulating technique, has cooling channels 10 incorporated in it to keep the positron emission tomography detection units 5 at a constant working temperature. An optimal cooling effect is achieved if the encapsulating material has a high thermal conductivity, for example by mixing in filling materials with a high thermal conductivity such as aluminum oxide, aluminum nitride, boron nitride, silicon carbide or quartz.

The second example embodiment can be adapted or refined by arranging cooling elements in the gap 6 itself. For example, cooling mats (for example, in the form of a water cooling) can be incorporated, in order to thus keep the heat of the magnetic resonance imaging gradient coil 2 away from the carrier tube 7 and the positron emission tomography detection unit 5.

Another possibility for reducing the noise, vibration or heat transmission from the magnetic resonance imaging gradient coil 2 to the carrier tube 7 is to evacuate the gap 6.

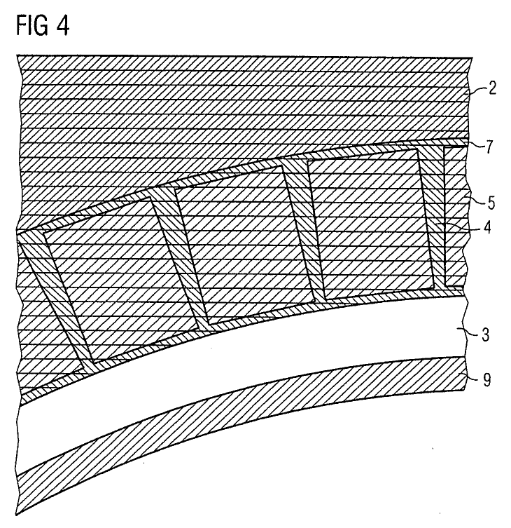

FIG. 4 shows a device for superposed magnetic resonance imaging and positron emission tomography imaging according to the third example embodiment of the present invention.

In the third example embodiment, the carrier tube 7 is manufactured by a vacuum molding, die casting or injection molding method, or a combination of these two methods. The carrier tube 7 and the magnetic resonance imaging gradient coil 2 are preferably formed integrally. The advantages thereof are a further reduction in the spatial requirements and/or an integration of further cooling layers. Furthermore, this stiffens the overall structure, leading to a decrease in noise.

The invention is not limited by the disclosed example embodiments, but modifications and equivalent embodiments are possible within the scope of the invention, which is defined by the claims.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A device for superposed magnetic resonance imaging and positron emission tomography imaging, comprising:
a magnetic resonance imaging magnet which defines a longitudinal axis;
a magnetic resonance imaging gradient coil arranged radially within the magnetic resonance imaging magnet;
a magnetic resonance imaging RF coil arranged radially within the magnetic resonance imaging gradient coil; and
a plurality of positron emission tomography detection units arranged in pairs opposite one another about the longitudinal axis, the plurality of positron emission tomography detection units being arranged radially within the magnetic resonance imaging gradient coil, being insertable into the device and being removable from the device along the longitudinal axis.

2. The device as claimed in claim 1, further comprising:
a carrier tube, including a multiplicity of pockets which each extend along the longitudinal axis, to accommodate at least one positron emission tomography detection unit.

3. The device as claimed in claim 2, further comprising:
at least one cooling channel to accommodate a cooling medium, provided between two adjacent pockets.

4. The device as claimed in claim 2, wherein the pockets include a metal layer on an inner face of the pockets.

5. The device as claimed in claim 2, wherein
cooling elements are arranged in a gap, present between the carrier tube and the magnetic resonance imaging gradient coil.

6. The device as claimed in claim 2, wherein
an evacuated gap is present between the carrier tube and the magnetic resonance imaging gradient coil.

7. The device as claimed in claim 2, wherein
the carrier tube is produced by at least one of vacuum molding, die casting and injection molding.

8. The device as claimed in claim 7, wherein
the carrier tube and the magnetic resonance imaging gradient coil are formed integrally.

9. The device as claimed in claim 1, wherein
the positron emission tomography detection units each include an avalanche photodiode array with a lutetium oxyorthosilicate crystal array and an electrical amplifier circuit upstream thereof.

10. The device as claimed in claim 3, wherein the pockets include a metal layer on an inner face of the pockets.

11. The device as claimed in claim 3, wherein
cooling elements are arranged in a gap, present between the carrier tube and the magnetic resonance imaging gradient coil.

12. The device as claimed in claim 3, wherein
an evacuated gap is present between the carrier tube and the magnetic resonance imaging gradient coil.

13. The device as claimed in claim 5, wherein
an evacuated gap is present between the carrier tube and the magnetic resonance imaging gradient coil.

14. The device as claimed in claim 2, wherein
the positron emission tomography detection units each include an avalanche photodiode array with a lutetium oxyorthosilicate crystal array and an electrical amplifier circuit upstream thereof.

15. The device as claimed in claim 2, further comprising:
a carrier tube including a multiplicity of pockets, which each extend along the longitudinal axis, the pockets each accommodating at least one positron emission tomography detection unit.
